(19) United States
(12) Patent Application Publication
(10) Pub. No.: US 2010/0267090 A1
(43) Pub. Date: Oct. 21, 2010

(54) NOVEL SACCHARIDE PRIMER

(75) Inventor: Tomonori Sato, Yokohama (JP)

Correspondence Address: TAROLLI, SUNDHEIM, COVELL & TUMMINO L.L.P., 1300 EAST NINTH STREET, SUITE 1700, CLEVELAND, OH, 44114, US

(73) Assignee: GlycoMedics, Inc.

(21) Appl. No.: 12/757,084

(22) Filed: Apr. 9, 2010

Related U.S. Application Data
(62) Division of application No. 10/925,210, filed on Aug. 25, 2004, now abandoned.

(30) Foreign Application Priority Data
Oct. 14, 2003 (JP) 2003-354295

(51) Int. Cl. C12P 19/00

(52) U.S. Cl. 435/72

(57) ABSTRACT

The present invention discloses a saccharide primer for synthesizing, in culture cells, an O-glycan sugar chain having the structure of sugar chain-amino acid-alkyl group or alkyl group derivative.

### BACKGROUND OF THE INVENTION

1. Field of the Invention

The present invention relates to a method for producing an oligosaccharide used in pharmacology, medicine, sugar chips, and the like.

2. Background Information

Oligosaccharides, which play an important role in intercellular recognition or as receptors for viruses and the like, have the potential for applications in biotechnology and pharmaceuticals. In particular, construction of a saccharide library comprising various polysaccharides is thought to contribute to the development of biotechnology and pharmaceuticals. Conventionally oligosaccharides are obtained by extraction from natural resources, for example, from bovine brains, by organic synthesis, or by enzymatic synthesis with a prepared recombinant polysaccharide synthase. For the extraction of natural products, however, it was difficult to obtain the materials. In addition, organic synthesis is technically difficult, requiring tremendous amounts of time and cost. Currently, although enzymatic synthesis is widely used as a method for synthesizing oligosaccharides, this method is also costly and is not necessarily suitable for inexpensively obtaining many kinds of oligosaccharide in large quantities.

Oligosaccharides are present in vivo as glycolipids, glycoproteins, and polysaccharides, among which glycoproteins include O-glycan-type sugar chains and N-glycan-type sugar chains. Among these, polysaccharides have been easily obtainable. In addition, since, among glycoprotein sugar chains, the N-glycan-type sugar chain exists in large quantities in vivo, it has been obtained relatively easily.

In terms of obtaining sugar chains from glycolipids, the present inventors developed a method whereby oligosaccharides are produced by first administering a saccharide primer to animal cells and extending the sugar chain inside the cells. In addition, they have also reported on a saccharide primer for administering to cells and synthesizing an oligosaccharide inside the cells (see JP-2000-247992-A and D. J. Moloney et al., Nature, 406, 369-375 (2000)).

When a saccharide primer resulting from the linkage of an alkyl group, such as a dodecyl group, to a monosaccharide or a disaccharide (saccharide-alkyl group) is administered to culture cells, new sugar chains are extended at the tip of the saccharide primer by a glycosyltransferase that is present inside the cell and secreted from the cell. This has so far been utilized to obtain approximately 50 species of glycolipid type sugar chains, and a saccharide library has been thus constructed.

Among the sugar chains from glycoproteins, however, it was still difficult to obtain an O-glycan-type sugar chain.

It should be noted that the use of a compound having a linked sugar-amino acid structure as a substrate for a glycosyltransferase involved in the biosynthesis of O-glycans in a cell-free system has been reported (see D. J. Moloney et al., Nature, 406, 369-375 (2000)). However, this was not an attempt to obtain O-glycan sugar chains in large amounts using culture cells.

In addition, it has also been reported that Benzyl-GalNAc was administered to cells to obtain an O-glycan-type sugar chain (see J. P. Zanetta et al., Glycobiology, 10, 565-575, (2000) and V. Gouyer, Frontiers in Bioscience 6, 1235-1244, (2001)). According to this report, however, the introduction of Benzyl-GalNAc into the cells was difficult; in addition, it was necessary to add an organic solvent to the culture medium in order to dissolve the compound. This was not a condition that was appropriate for the cell culture. Furthermore, the synthesized O-glycan-type sugar chain accumulated inside the cells and was not released from the cells. Consequently, Benzyl-GalNAc was not effective as a saccharide primer.

### SUMMARY OF THE INVENTION

An object of the present invention is to prepare an O-glycan-type sugar chain by synthesizing an O-glycan among the glycoprotein-type sugar chains using a saccharide primer in cells.

Conventional saccharide primers had structures wherein a single-chain alkyl group is linked to a saccharide, such as a monosaccharide or a disaccharide. Extension reactions were observed for glycolipid type oligosaccharides, but extension reactions did not occur for glycoprotein-type sugar chains. It was necessary to obtain a set of glycolipid-type and glycoprotein-type oligosaccharides in order to build a saccharide library. Glycoproteins can be generally categorized into N-glycans and O-glycans due to the difference in the biosynthesis pathway; as N-glycans are expressed in large quantities in cells, they can easily be obtained by extraction from the cells. On the other hand, as O-glycans are expressed in small quantities, extraction from cells has been difficult. Consequently, given that among the intracellular glycoprotein-type sugar chains, the quantity of O-glycans expressed is small, it has been thought that it should be effective for building a library to make them in the cells using a saccharide primer method. Therefore, the present inventors designed a novel saccharide primer for making O-glycans.

In this study, a saccharide primer was synthesized to elongate an O-linked glycoprotein-type sugar chain and was administrated to culture cells, and a structural analysis was performed for the sugar chain obtained. A sugar-amino acid-type primer, in which threonine (Thr) as well as a dodecyl group was linked to N-acetylgalactosamine (GalNAc) (saccharide-amino acid-alkyl group), was chemically synthesized and administered to various animal cells. After a predetermined time, lipid components were extracted from the culture medium fraction, and structural analysis of the products was performed using HPTLC and MALDI-TOF MS/MS. As a result, a characteristic O-glycan core structure and an extension of oligosaccharides such as sialyl-Tn antigens were detected. In this way, an O-glycan-type sugar chain could be synthesized using the aforementioned saccharide primer, bringing the present invention to completion.

That is to say, the present invention is as follows:

(1] A saccharide primer for synthesizing an O-glycan-type sugar chain represented by sugar chain-amino acid-alkyl group or alkyl group derivative.

(2) The saccharide primer of (1) wherein the sugar chain is GalNAc.

(3) The saccharide primer of (1) wherein the amino acid is Ser or Thr.

(4) The saccharide primer of (1) wherein a CH₂—CH₂ bond in the alkyl group or the alkyl group derivative has been substituted by —S—S— or —NHCO— and represented by sugar chain-amino acid-alkyl group or alkyl group derivative-X.

(5) The saccharide primer of (1) wherein the alkyl group is —(CH₂)₁₂.

(6) The saccharide primer of (1), wherein a functional group selected from the group consisting of —N₃, —NH₂, —OH, —SH, —COOH, —OC(O)CH═CH₂, and —CH═CH₂ is further linked to the alkyl group and represented by sugar chain-amino acid-alkyl group or alkyl group derivative.

(7) A compound represented by Formula (I): (G₁)ₓ(G₂)_{y}(G₃)_{z}-Aₘ-L-X (in the formula, G₁, G₂, and G₃ are independent monosaccharide residues with a pyranose ring or derivatives thereof; (G₁)ₓ(G₂)_{y}(G₃)_{z} is linear or branched; Aₘ is a sequence of 1 to 5 amino acids or derivatives thereof, and when there are a plurality of amino acids, the constituent amino acids may be identical or different; L is a linking group selected from the group consisting of —O—R—, —S—R—, —NH—R—, and derivatives thereof, R being an alkyl group, the main carbon chain thereof consisting of 6 to 20 carbons, or a derivative thereof; X is either not present or a functional group selected from the group consisting of —N₃, —NH₂, —OH, —SH, —COOH, —OC(O)CH═CH₂, and —CH═CH₂; x, y, and z are independent integral numbers between 0 and 10. However, all of x, y, and z cannot be simultaneously 0).

(8) The compound of (7) wherein (G₁)ₓ(G₂)_{y}(G₃)_{z} is GalNAc.

(9) The compound of (7) wherein Aₘ is Ser or Thr.

(10) The compound of (7), wherein the alkyl group derivative is a derivative in which a CH₂—CH₂ bond in the alkyl group is substituted by —S—S— or —NHCO—.

(11) The compound of (7) wherein L is —O—(CH₂)₁₂.

(12) The compound of (7) wherein X is —N₃.

(13) A compound which is GalNAcα1-Ser-O—(CH₂)ₙ—N₃ or GalNAcα1-Thr-O—(CH₂)ₙ—N₃ (wherein, n is from 4 to 20).

(14) The compound of (13) wherein n is 12.

(15) The compound of (7), which is a saccharide primer.

(16) A method for synthesizing an O-glycan-type sugar chain inside a cultured cell, comprising adding the saccharide primer of (1) to a cultured cell.

(17) The method of claim 16 using a cell cultured using a high-density culture method.

(18) The method of (16) wherein the cell is selected from the group consisting of an animal cell, a plant cell, an insect cell, and yeast.

(19) The method of (18) wherein the cell is an animal cell.

(20) The method of (19) wherein the cell is a human cell.

(21) The method of (16) wherein the cell contains a vector in which a DNA coding for a glycosyltransferase has been integrated.

(22) The compound synthesized by the method of claim 16 wherein the sugar chain has the structure of an O-glycan-type sugar chain-amino acid-alkyl group or alkyl group derivative-X, X being a functional group selected from the group consisting of —N₃, —NH₂, —OH, —SH, —COOH, —OC(O)CH═CH₂, and —CH═CH₂.

(23) A compound represented by Formula (II): GC-Aₘ-L-X (in the formula, GC is an O-glycan-type sugar chain; Aₘ is a sequence of 1 to 5 amino acids or derivatives thereof, and when there are a plurality of amino acids, the constituent amino acids may be identical or different; L is a linking group selected from the group consisting of —O—R—, —S—R—, —NH—R—, and derivatives thereof, R being an alkyl group, the main carbon chain thereof consisting of 6 to 24 carbons or a derivative thereof; X is either not present or a functional group selected from the group consisting of —N₃, —NH₂, —OH, —SH, —COOH, —OC(O)CH═CH₂, and —CH═CH₂; x, y, and z are independent integral numbers between 0 and 10. However, all of x, y, and z cannot be simultaneously 0).

(24) The compound of (23) wherein Aₘ is Ser or Thr.

(25) The compound of (23) wherein the alkyl group derivative is a derivative in which a CH₂—CH₂ bonds in the alkyl group is substituted by —S—S— or —NHCO—.

(26) The compound of (23) wherein L is —O—(CH₂)₁₂

(27) The compound of (23) wherein X is —N₃.

(28) The compound of (23) wherein GC is selected from the group consisting of Galβ1-3GalNAc, Galβ1-3(GlcNAcβ1-6)GalNAc, GlcNAcβ1-3GalNAc, GlcNAcβ1-3(GlcNAcβ1-6)GalNAc, GalNAcα1-3GalNAc, GlcNAcβ1-6GalNAc, GalNAcα1-6GalNAc, and Galα1-3GalNAc, or a derivative thereof.

(29) A sugar chip containing the compound of (23).

As shown in the examples described in the present specification, it was possible to synthesize an O-glycan-type sugar chain in cells by introducing a saccharide primer of the present application in the cells. A variety of O-glycan-type sugar chains can be synthesized by varying the combination of saccharide primers and cells, allowing a saccharide library to be constructed, and the obtained saccharide library can be immobilized on a solid phase to manufacture characteristic sugar chips that are suited to a variety of objectives.

### BRIEF DESCRIPTION OF THE DRAWINGS

FIG. 1 shows the results of the analysis of an MNK45 cell culture medium fraction by HPTLC. Stained with Rsolsinol/HCl.

FIG. 2 shows an MS spectrum of product A2 from an MKN45 cell. These are the results of measurement in a negative ion mode of a sialylation product A2 obtained by administering a GalNAc-Thr-C12 primer to an MKN45 cell.

FIG. 3 shows a MALDI-PSD spectrum of product A2 from a MKN45 cell.

FIG. 4 shows an MS spectrum of product A1 from a MKN45 cell. These are the results of measurement in a negative ion mode of a sialylation product A1 obtained by administering the GalNAc-Thr-C12 primer to an MKN45 cell.

FIG. 5 shows a MALDI-PSD spectrum of product A1 from a MKN45 cell.

FIG. 6 shows the results of an analysis of a HuH7 cell culture medium fraction by HPTLC. Stained with Rsolsinol/HCl.

FIG. 7 shows an MS spectrum of product A1 from a HuH7 cell. These are the results of measurement in a negative ion mode of a sialylation product A1 obtained by administering a GalNAc-Thr-C12 primer to an HuH7 cell;

FIG. 8 shows a MALDI-PSD spectrum of product A1 from a HuH7 cell.

FIG. 9 shows a MS spectrum of product A2 from a HuH7 cell. These are the results of measurement in a negative ion mode of a sialylation product A2 obtained by administering a GalNAc-Thr-C12 primer to an HuH7 cell.

FIG. 10 shows a MALDI-PSD spectrum of product A2 from a HuH7 cell.

FIG. 11 shows core structures of O-linkage-type sugar chains.

FIG. 12 shows a sugar chain elongation pathway when Benzyl-GalNAc is administered to an HT-29 cell.

### DETAILED DESCRIPTION

In the following, the present invention will be described in detail.

The present invention is a saccharide primer which can be used for biosynthesizing a sugar chain in a cell, having a sugar chain-amino acid-alkyl group or alkyl group derivative structure, and is indicated by Formula (I): (G₁)ₓ(G₂)_{y}(G₃)_{z}-Aₘ-L-X (in the formula, G₁, G₂, and G₃ are independent monosaccharide residues with a pyranose ring or derivatives thereof; (G₁)ₓ(G₂)_{y}(G₃)_{z} may be linear or branched; Aₘ is a sequence of 1 to 10, preferably 1 to 5, more preferably 1 or 2 amino acids or derivatives thereof, and particularly preferably 1 amino acid or derivative thereof, and when there are a plurality of amino acids, the constituent amino acids may be identical or different; L is a linking group selected from the group consisting of —O—R—, —S—R—, —NH—R—, and derivatives thereof, R being an alkyl group or a derivative thereof; X is either not present or a functional group selected from the group consisting of —N₃, —NH₂, —OH, —SH, —COOH, —OC(O)CH═CH₂, and —CH═CH₂; x, y, and z are independent integral numbers between 0 and 10. However, all of x, y, and z cannot be simultaneously 0).

In the compound of the present invention of Formula (I):, G₁, G₂, and G₃ are independent monosaccharide residues with a pyranose ring or derivatives thereof. Any monosaccharide may be used for the monosaccharides, including N-acetylgalactosamine (GalNAc), N-acetylglucosamine (GlcNAc), xylose (Xyl), galactose, glucose, arabinose, mannose, L-fucose (Fuc), sialic acid (Sia), and the like, among which N-acetylgalactosamine (GalNAc), D-xylose, D- or L-galactose, D-glucose, D- or L-arabinose, D-mannose, L-fucose (Fuc), and the like are preferred for G₃. Among them, in particular, N-acetylgalactosamine, fucose, or xylose is preferred.

Examples of (G₁)ₓ(G₂)_{y}(G₃)_{z} include GalNAc, Fuc, Xyl, Galβ1-3GalNAc, Galβ1-3(GlcNAcβ1-6)GalNAc, GlcNAcβ1-3GalNAc, GlcNAcβ1-3(GlcNAcβ1-6)GalNAc, GalNAcα1-3GalNAc, GlcNAcβ1-6GalNAc, GalNAcα1-6GalNAc, Galα1-3GalNAc, and the like; however, it is not limited thereto.

There are likewise no restrictions on the amino acid, and any amino acid can be used; furthermore, a derivative thereof can also be used. Preferably, this is threonine, serine-hydroxylysine, hydroxyproline, or hydroxylysine, among which threonine or serine is preferred. There are no restrictions on the amino acid derivative, and for instance, RCH(NH₂)CO—, RCH(NH₂)CO₂₁—, RCH(NH₂)CONH₂, RCH(NH₂)CH₂OH, RCH(NH₂)CHO, and RCH(CO₂H)NH— can be considered as derivatives for an amino acid represented by RCH(NH₂)COOH.

In the compound of Formula (I), L is a linking group selected from the group consisting of —O—R—, —S—R—, —NH—R—, and derivatives thereof, -L- being preferably —O—R—. Here, R is an alkyl group represented by (CH₂)ₙ, a derivative of the alkyl group wherein some hydrogen atoms have been substituted, or an alkyl group derivative in which the alkyl group comprises bonds, such as —S—S—, NHCO—, or the like, that is to say, some of the CH₂—CH₂ bonds in the alkyl group have been substituted by —S—S—, —NHCO—, or the like, or a hydrophobic group having the same hydrophobicity as the alkyl group or a derivative thereof, in which the number of carbon atoms n in the main carbon chain is an integral number between 4 and 24, preferably between 6 and 18, and particularly preferably 12 (a dodecyl group in case R is an alkyl group represented by (CH₂)ₙ). If n is less than 6 or more than 24, even if the compound of Formula (I) is provided to a cell as a saccharide primer, the sugar-adding capability of the cell to the saccharide primer is low. Whether the saccharide primer of the present invention is introduced into the cell and sugar is added depends on the balance between the hydrophilic groups and the hydrophobic groups of the saccharide primer. Consequently, as long as the balance between the hydrophilic groups and the hydrophobic groups of the saccharide primer is not greatly different from that in the case of the alkyl group where R is represented by (CH₂)ₙ, R may be an alkyl group derivative wherein some of the hydrogen atoms have been substituted by —N₃, —NH₂, —OH, —SH, —COOH, —OC(O)CH═CH₂, —CH═CH₂, or the like. In addition, as mentioned above, some of the CH₂—CH₂ bonds in R may be substituted by —S—S—, —NHCO—, or the like. Furthermore, since the overall balance between the hydrophilicity and the hydrophobicity of the saccharide primer does not vary largely as long as it has the same hydrophobicity as the alkyl group, R may be any hydrophobic group having the same hydrophobicity as the alkyl group represented by (CH₂)ₙ. The balance between the hydrophilicity and the hydrophobicity of a molecule can be predicted using, for instance, ChemDraw (CambridgeSoft) and would show log P values of between 3 and 8 for the other portion than the sugar chain in the saccharide primer.

In the compound of Formula (I), X is a group selected from —N₃, —NH₂, —OH, —SH, —COOH, —OC(O)CH═CH₂, and —CH═CH₂. Among these, X is preferably —N₃ or —NH₂, and more preferably —N₃. X is a functional group for immobilization when the sugar chain is to be immobilized onto a solid phase. In addition, by linking X, the saccharide primer inside the cell become more resistant to degradation so that a sugar chain can be synthesized more effectively than when X is absent.

Examples of the saccharide primer of the present invention include GalNAcα1-Ser-(CH₂)₁₂—N₃ and GalNAcα1-Thr-(CH₂)₁₂—N₃; however, it is not limited thereto.

The present invention also includes a method for preparing the saccharide primer represented by the aforementioned General Formula (I).

The saccharide primer of the present invention can be synthesized as follows:

The sugar chain represented by (G₁)ₓ(G₂)_{y}(G₃)_{z} in the saccharide primer indicated by the General Formula (I) can by synthesized by the methods described in the following literature:

T. Murata, T. Usui, Trends in Glycoscience and Glycotechnology, 12, No. 65, 161-174 (2000)

J. Tamura, Trends in Glycoscience and Glycotechnology, 13, No. 69, 65-68 (2001)

M. Ujita, M. Fukuda, Trends in Glycoscience and Glycotechnology, 13, 70, 177-191 (2001)

In addition, linkage between the sugar chain and an amino acid can be performed by the methods described in the following literature:

H. K. Ishida, H. Ishida, M. Kiso, and A. Hasegawa, Tetrahedron Asymmetry, 5, 2493-2512 (1994)

T. Inazu, Hide-ki Ishida, R. Nagano, K. Tanaka, and K. Haneda, “Peptide Science 1999: Proceedings of the 36th Japanese Peptide Symposium” ed. by H. Aoyagi, The Japanese Peptide Society, pp. 121-124 (2000)

T. Inazu, M. Mizuno, T. Yamazaki, and K. Haneda, “Peptide Science 1998: Proceedings of the 35th Symposium on Peptide Science,” ed. by M. Kondo, Protein Research Foundation Osaka, pp. 153-156 (1999)

Examples of the cells that may be used for preparing oligosaccharides using the saccharide primer of the present invention include eukaryotic cells having genes that are involved in saccharide synthesis, including mammalian cells, insect cells, plant cells, yeast, and the like. Examples of animal cells include cells derived form various animals, normal cells derived from animal tissues, animal cancer cells, animal diploid fibroblasts, animal vascular endothelial cells, and the like, but cells derived from humans are preferred. Synthesizing large amounts of sugar chains requires an established cell line, which allows for culturing over generations. Established cell lines express their characteristic saccharide synthesis, and appropriate selection of a cell line allows the set of O-glycan sugar chains expressed by the cells to be obtained. In addition, almost all the sugar chain biosynthetic pathways can be covered by using a multiplicity of cells, allowing a complete saccharide library to be constructed. Examples include the MKN45 cell, which is a human gastric cancer cell, and the HuH7 cell, which is a human hepatocellular carcinoma cell.

Various species of sugar chains can be obtained by combination of the species of saccharide primer and the cell type.

In addition, by either activating or inhibiting a specific sugar chain biosynthetic pathway in these cells, cells expressing any sugar chain biosynthetic pathway can be obtained, and the desired sugar chain can be synthesized. For instance, cells expressing any sugar chain biosynthetic pathway can be created by introducing or deleting DNA coding for glycosyltransferase that participates in a specific sugar chain biosynthetic pathway to the cells. Alternatively, an inhibitor of enzyme that participates in a specific sugar chain biosynthetic pathway can also be administrated to the cells. These gene manipulations can be performed according to methods described in literature well-known to those skilled in the art, such as J. Sambrook, E. F. Fritsch, and T. Maniatis (1989): Molecular Cloning, A Laboratory Manual, second edition, Cold Spring Harbor Laboratory Press, and Ed Harlow and David Lanc (1988): Antibodies, A Laboratory Manual, Cold Spring Harbor Laboratory Press. They can be performed based on genetic engineering techniques; for instance, it suffices to integrate DNA coding for glycosyltransferase into an adequate expression vector and introduce the resulting expression vector into cells.

In addition, prokaryotic cells that do not have a sugar chain biosynthetic pathway can be made to synthesize an O-glycan-type sugar chain by introducing DNA coding for glycosyltransferase that participates in a sugar chain biosynthetic pathway of eukaryotic cells into the prokaryotic cells by a genetic engineering method. A sugar chain can be synthesized by this method, using cells that are used broadly in genetic engineering, such as Escherichia coli and Bacillus subtilis.

In order to synthesize large amounts of sugar chains using the saccharide primer of the present invention, cells must be cultured on a large scale. Large-scale culture can be achieved by high-density culture, in which cells are cultured at a high density. For high-density culture, the microcarrier-culture method, culture with culture layers on a cell immobilization disc, culture system using a hollow fiber module, suspension culture of free cells, a method using multi-step culture apparatus or a roller bottle, or a method wherein cells are cultured by being immobilized onto a microcapsule and the like are available, but the use of the microcarrier culture method, culture apparatus using cell immobilization disk, a culture system using hollow fiber module, or a method using a suspension culture of free cells is preferred.

In the microcarrier, a matrix, such as collagen, gelatin, cellulose, crosslinking dextran, or a synthetic resin, such as polystyrene, or the use of charged groups, such as dimethylaminopropyl, dimethylaminoethyl, trimethylhydroxyaminopropyl, and a group to which a negative charge has been added, is preferably used. In addition, matrices coated with collagen or gelatin are also used. Commercially available products include Cytodex-1 (Pharmacia) and Cytodex-3 (Pharmacia), in which dimethylaminoethyl has been added to a crosslinking dextran. Examples of hollow fibers include those in which modified cellulose has been used (Vitafiber, Amicon).

A method for preparing microcapsules is known, in which cells are embedded inside by using collagen that forms a water-permeable gel or sodium alginate (A. Klausner, Bio/Technol., 1, 736 (1983)).

Small-scale cultures of microcarriers start by introducing PBS(-) containing microcarriers into a spinner flask, sterilizing with vapor at high pressure, exchanging the solution with a culture medium, and inoculating cells. The medium is exchanged at appropriate intervals, and after cells have proliferated to be confluent on the cell microcarrier, the saccharide primer is administered. For cells requiring a growth factor for proliferation and survival, human vascular endothelial cells, vascular endothelial growth factor (VEGF), fibroblast growth factor (FGF), and the like are added to the culture medium.

The microcarrier culture has the advantages that a number of cells equivalent to 100 plates with an internal diameter of 100 mm is obtained with one 200 ml-scale culture bottle; furthermore, as the culture is a high-density culture with 4 times the number of cells per liquid volume unit, the amount of oligosaccharide primer introduced is also low; in addition, novel oligosaccharides can be detected, which cannot be identified with plated cells.

In order to have culture cells synthesize an O-glucan-type sugar chain, one μM to several hundred μM, and preferably 10 to 100 μM, of the saccharide primer of the present invention is introduced into cells that have proliferated to be confluent, using a serum-free or low-serum culture medium, and these are cultured for 1 to 5 days at 37° C. The cells incorporate the saccharide primer, in the intracellular Golgi apparatus inside the cells, sugars are further added to the sugar chain portion of the saccharide primer by the sugar chain biosynthetic pathway that the cells possess, and the product of sugar addition is excreted to the outside of the cells. In this way, a product stock solution containing the elongated sugar chain can be obtained. The supernatant of the culture medium is collected, and concentration, separation, and structural analyses are performed, and a library of several species of oligosaccharides is obtained. As the species and introduction quantity of the saccharide primer, the culture medium, and the number of culture days differ depending on the cell species, finding the optimum culture conditions for each cell leads to efficient production of oligosaccharides.

The oligosaccharide contained in the harvested solution is concentrated and separated using affinity chromatography, ultrafiltration, or ammonium sulfate precipitation and the like, and its structure is analyzed by high-performance thin layer chromatography (HPTLC), MALDI-TOF MS, NMR, or the like. Regarding unknown substances, after blotting with high-performance thin layer chromatography and treating with an enzyme, its structure is inferred from analysis of the composition of the substance obtained.

The present invention also includes the saccharide primer to which a sugar has been added, which has been obtained in this manner; that is to say, the compound represented by elongated sugar chain-amino acid-alkyl group. The compound is represented by the General Formula (II) GC-Aₘ-L-X (in the formula, GC is an O-glycan-type sugar chain elongated by the addition of a sugar to the saccharide primer used; the definitions of Aₘ, L, R, and X are equivalent to those described above).

Herein, GC includes any O-glycan-type sugar chains that are found in the natural world and is classified from core type 1 to core type 8, according to the structure of the saccharide that extends from the GalNAc residue as follows:

Core type 1: Galβ1-3GalNAc; core type 2: Galβ1-3(GlcNAcβ1-6)GalNAc; core type 3: GlcNAcβ1-3GalNAc; core type 4: GlcNAcβ1-3(GlcNAcβ1-6)GalNAc; core type 5: GalNAcα1-3GalNAc; core type 6: GlcNAcβ1-6GalNAc; core type 7: GalNAcα1-6GalNAc; and core type 8: Galα1-3GalNAc.

GC in the General Formula indicated above is a derivative having a structure wherein other sugars are further linked at position 3 and position 6 of these core-type sugars.

The sugar chain obtained can be used in various applications according to the added sugar chain.

A saccharide library set is obtained by collecting together O-glycan-type sugar chains prepared using the saccharide primer of the present invention and/or sugar chains from glycolipids, sugar chains from polysaccharides, and N-glycan-type sugar chains obtained by other methods. The saccharide library set may be derived from a certain cell, may be derived from a certain animal species, or may include any sugar chain that exists in the natural world and may be any combination of partial sugar chains thereof. A sugar chip containing the required sugar chains can be obtained by immobilizing the set of these saccharide libraries onto a solid phase. A sugar chip enables comprehensive analysis of the interaction between a sugar chain and a protein that only exists in tiny amounts in the cell or a gene.

To prepare a sugar chip, the compound having a sugar chain represented above by General Formula (II) GC-Aₘ-L-X is aligned and linked onto a solid phase for immobilization. Here, linking can be facilitated by a coupling reaction or the like by introducing a functional group on the solid phase, such as an amino group or a carboxyl group that may bind covalently to X. A nitrocellulose membrane, a nylon membrane, a glass plate, or a resin plate, such as those made of polystyrene or polycarbonate, can be used as the solid phase.

There are likewise no restrictions on the alignment method, and any method may be used as long as the method allows the aforementioned compound to be aligned at high densities on the solid phase. For instance, an arrayer may be used, which spots the compound solution onto the solid phase. A variety of systems are available for the spotting arrayer, such as pins, feather pens, inkjets, capillaries, pin and ring, and the like, and any system can be used. In addition, a picking robot may also be used.

The present invention also includes a sugar chip obtained in this manner wherein a compound having an O-glycan-type sugar chain represented by the General Formula (II) GC-Am-L-X has been immobilized.

Furthermore, in the General Formula (II) GC-Aₘ-L-X, X may be absent. If X is not present in the saccharide primer to be used, the compound represented by GC-Aₘ-L is obtained. In addition, X may also be excised and removed from a compound wherein X is a group selected from —N₃, —NH₂, —OH, —SH, —COOH, —OC(O)CH═CH₂, and —CH═CH₂ by well-known methods.

### EXAMPLES

In the following, the present invention will be described in more concrete terms by way of examples; however, the present invention is not limited to these examples.

### Example 1

### Synthesis of GalNAc-Thr-C12 primer

1. Preparation of the reagents

NMP

Activated molecular sieve (4A, pellet form) was added to 500 ml of N-Methyl-2-pyrrolidone (NMP) (Kokusan Kagaku) and conserved at room temperature.

1 M Dimethylphosphinothyl chloride (Mpt-Cl)

Five mmol of Mpt-Cl (Tokyo Kasei) was placed in a 5 ml measuring flask, which was filled up with NMP, capped, covered with Parafilm, and conserved at 4° C.

Dimethylphosphinothioic Mixed Anhydride (Mpt-MA)

0.22 mmol of Fmoc-Thr(GalNAc)-OH or Lauric acid (Aldrich) and 3 ml of NMP were placed in a round-bottomed flask, and a calcium chloride tube was connected. The flask was placed in an ice bath and stirred with a stirrer for 3 minutes. After stirring, 150 μl of 2.0 M N,N-Diisopropylethylamine/N-Methylpyrrolidone (DIEA) (Applied Biosystems) was added, and after briefly stirring with a stirrer, 300 μl of the prepared 1 M Mpt-Cl was added. After stirring for 30 minutes on the ice bath, 150 μl of DIEA was added, which was stirred for some time.

Cleavage Mixture (for 0.1-1.5 g Peptide-Resin)

Using a chemical hood, 500 μl of ultra-pure water and 9.5 ml of Trifluoroaceticacid (TFA) (Applied Biosystems) were placed in an Erlenmeyer and mixed well by shaking.

20% (v/v) Piperidine/NMP

A volume of 400 m NMP and 100 ml of Piperidine (Wako) were paced in a light-tight glass container and conserved at room temperature.

2. Synthesis

0.22 mmol of Rink Amide MBHA Resin (Nova Biochem) was introduced in a column for solid phase peptide synthesis (Tokyo Rika Kikai Co.) and connected to a multiple solid phase synthesizer (Kokusan Kagaku). A volume of 6 ml of NMP was added thereto, and after shaking for 20 minutes, NMP was removed from the bottom of the column with an aspirator.

6 ml of 20% (v/v) Piperidine/NMP was poured into a column, and after shaking for 3 minutes, 20% (v/v) Piperidine/NMP was removed with an aspirator. This operation was repeated one more time. Then, 6 ml of 20% (v/v) Piperidine/NMP was poured in, shaken for 20 minutes, and removed with an aspirator.

Next, the operation of pouring 6 ml of NMP into the column, shaking for 1 minute, and removing with an aspirator was repeated 6 times.

After adding the prepared Mpt-MA (Fmoc-Thr(GalNAc)-OH) to the column and shaking for 1 hour, several resin particles were taken to another container with a Pasteur pipette, and reagents for Kaiser test Kokusan Kagaku) were used to perform a Kaiser test to examine the reaction efficiency.

After confirming that the amino acid residues were introduced with a high efficiency, the reaction solution was removed with an aspirator, and the operation of pouring 6 ml of NMP into the column, shaking for 1 minute, and removing with an aspirator was repeated 6 times.

The operation of deprotection, washing, performing a coupling reaction with Mpt-MA (Lauric acid), and washing was performed in the same way.

The column was removed from the multiple solid phase synthesizer, and a two-way stopcock (Tokyo Rika Kikai Co.) was connected. The cleavage mixture was added, and after stirring for 3 hours with a stirrer, the stopcock was opened to transfer the reaction solution to a round-bottomed flask. The reaction solution remaining on the inner wall of the column was also washed away with TFA and transferred to the flask. The two-way stopcock on the column was closed, a small amount of TFA was put into the column; this was stirred with a stirrer, the stopcock was opened, and the solution was transferred to the aforementioned round-bottomed flask (this operation was performed for a total of 3 times). TEA inside the round-bottomed flask was evaporated with an evaporator. The remaining liquid was transferred to a 50 ml centrifugation tube and dried with a lyophilizes. After adding N,N-Dimethylformamide (Wako) to the dried sample so as to obtain 10 mg/ml, this was passed through a membrane filter (0.45 μm), and purification by HPLC was performed with this as the sample. 17.4 mg (35 μmol) of the target compound was obtained. This was dissolved with dimethylsulphoxide (DMSO) (Sigma), so as to obtain 50 mM, which was used as the primer stock solution.

### Example 2

### 1. Structural Analysis of Products of Sialylation in MKN45 Cells

All the intracellular sugar chain elongation reactions were performed in a serum-free culture medium that did not contain phenol red. For MKN45 cells, RPMI1640 (11835-030, Invitrogen) was selected as the serum-free culture medium for the sugar chain elongation reaction and used by adding 5 mg/l of transferrin (holo bovine, Wako Pure Chemical), 5 mg/l of insulin (human, Sigma), and 30 nM selenium dioxide.

When sugar chain elongation was to be performed using a small amount, a 100 mmf dish was used, and when sugar chain elongation was performed using a large amount, a 200 ml spinner-bottle was used, culturing by stirring with a magnetic stirrer.

The sugar chain elongation reaction was performed as follows. Cells were recovered from the culture medium by centrifugation, washed with PBS(-) (Nissui Pharmaceutical), and then washed again with the serum-free culture medium for the sugar chain elongation reaction. These cells were stained by trypan blue and prepared at 1.5×10⁶ cells/ml, and then were interacted with the saccharide primer by resuspending them in a serum-free RPMI1640 culture medium containing 50 μM of primer.

The cells that were interacted with the primer were cultured for 48 hours, then left on ice or at 4° C. to stop the reaction. After recovering the culture medium from the reaction container, the cells were recovered while washing with PBS(-). The recovered cell suspension was centrifuged, the precipitated cells were recovered as the cell fraction, and the supernatant was recovered as the culture medium fraction. Note that, if experimental circumstances required quantitation of protein, the recovered cell fraction was resuspended in 500 μl PBS(-), of which 50 μl was taken for protein quantitation. In this case, the suspension was centrifuged again to recover the precipitate as the cell fraction, and the supernatant was added to the culture medium fraction.

Extraction from the cell fraction was performed by adding 1 ml of chloroform/methanol (C/M)=2/1 (v/v) and sonicating for 30 minutes.

The medium fraction was subjected to reverse-phase column chromatography to adsorb the lipids to the carrier, then extraction was performed. Sep-Pak C18 Plus (Waters) was used for small scales, and for large-scale extraction, an open column filled with Preparative C18 125 Å (Waters), which is the Sep-Pak C18 Plus carrier, was prepared. The column to which the medium fraction was adsorbed was washed with an amount of 10 times the bed volume of MilliQ water, then eluted with a quantity of 5 times the bed volume of a methanol/water solvent mixture (shown in results and discussion). After elution, the solvent was evaporated, and the pellet was conserved at 4° C. The pellet was dissolved again in a chloroform/methanol/water (C/M/W) solvent mixture for use.

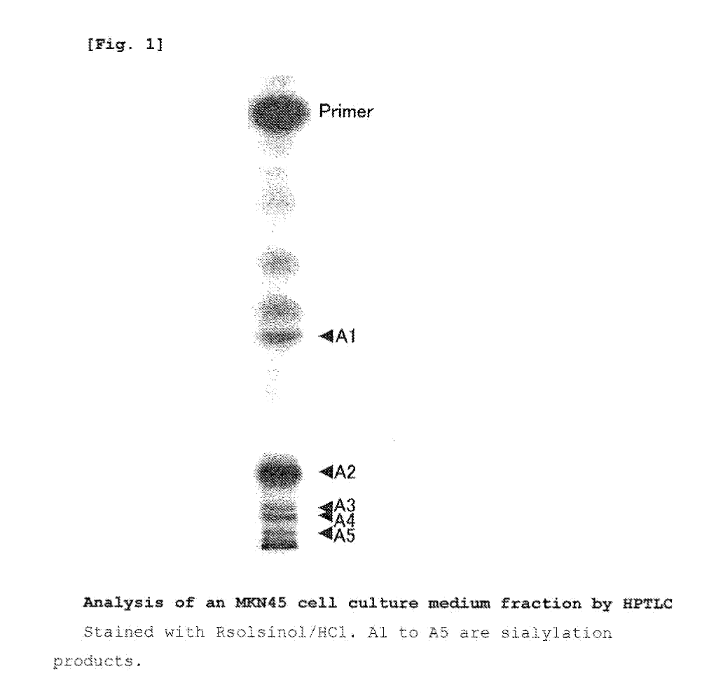

Lipids extracted from the cell fraction and the lipid fraction were separated using a high-performance thin layer chromatography (HPTLC) plate (Silicagel 60, Merck). A solvent mixture of chloroform/methanol/0.2% CaCl₂ aqueous solution (5/4/1) suited to the experimental system was used as the development system. For acidic glycolipids, bands were visualized in blue-violet color by spraying resorcinol-hydrochloric acid reagent and heating at 95° C., then analyzed at a wavelength of 580 nm using a densitometer (CS-9300PC, Shimazu). For neutral glycolipids, bands were visualized in red-violet color by spraying orcinol-sulfuric acid reagent and heating at 105° C., then analyzed at a wavelength of 540 nm using a densitometer. As a result, 5 bands of sialylation products were obtained (FIG. 1). The 5 sialylation products were respectively designated A1 to A5.

Analysis of Compound A2

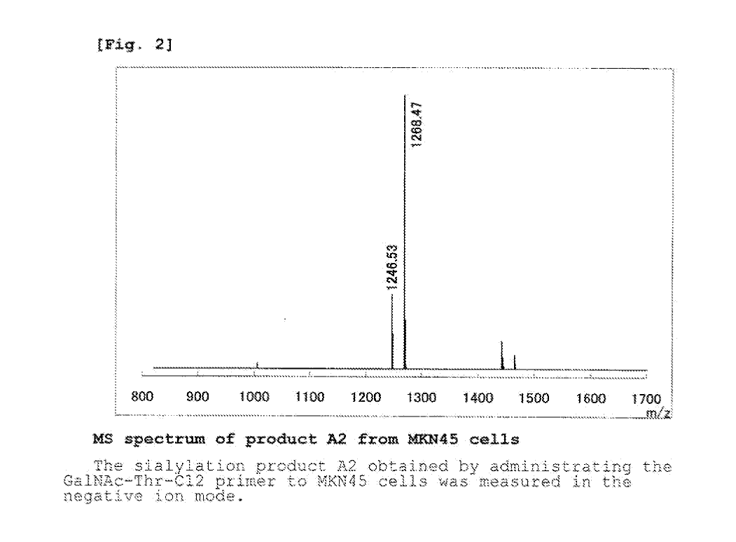

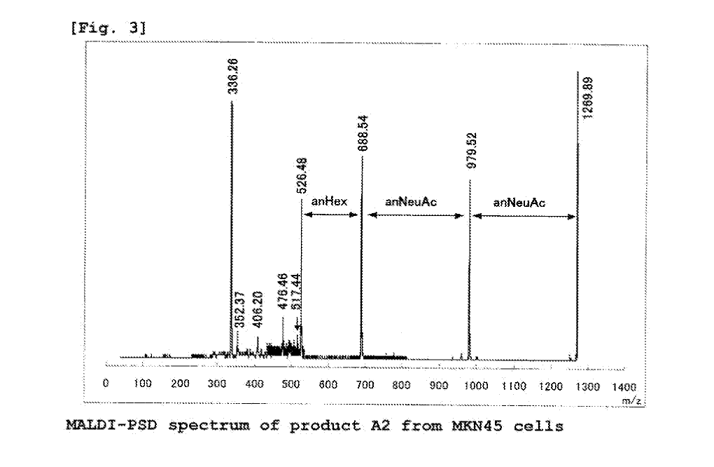

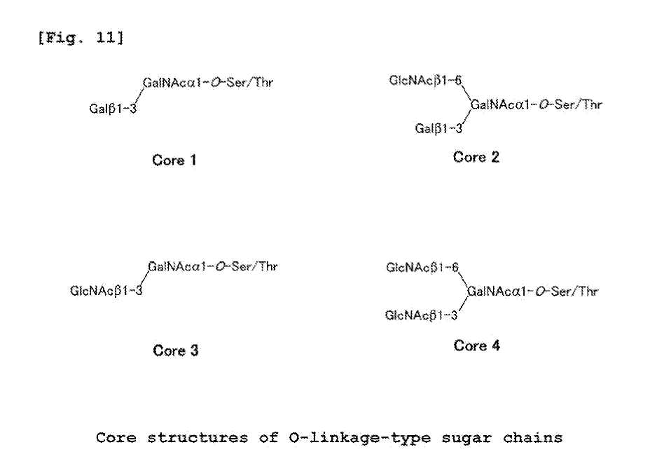

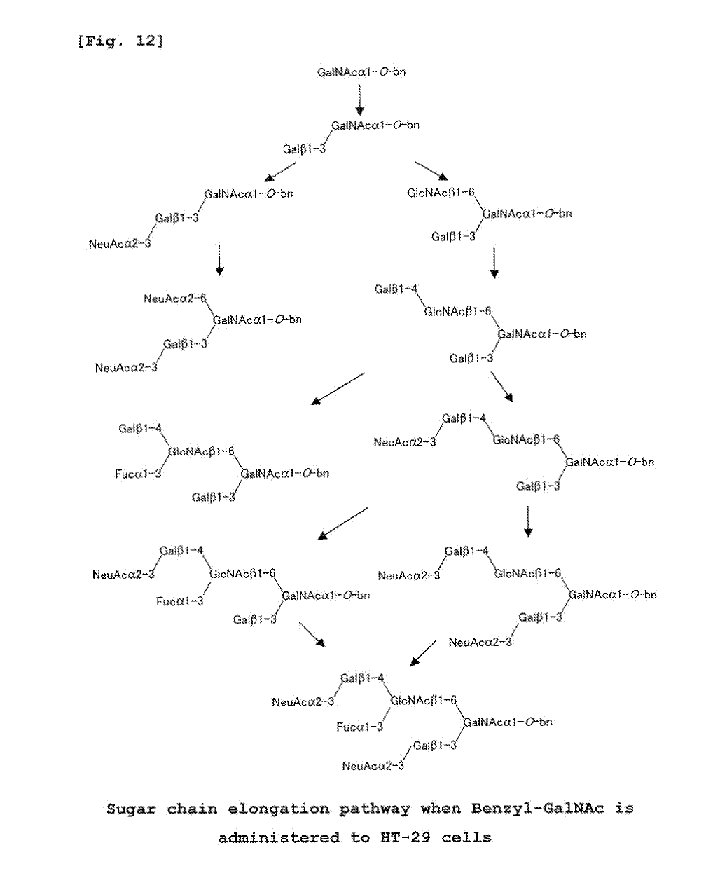

When measured in the negative ion mode, a peak was obtained at m/z=1268.47 (FIG. 2). Next, MS/MS measurement was performed with this molecule as the precursor ion (FIG. 3 and Table 1). The PSD spectrum revealed that this molecule had a structure wherein one Hex and two NeuAc were linked to the primer. Supposing that this is an O-linked-type sugar chain, it is anticipated that Hex is Gal and that Gal is linked to GalNAc via a β1-3 linkage (Core 1) (FIG. 11). In addition, from the fact that peaks are observed at m/z=517.44 and 476.35, each of the two NeuAc is believed to be linked to Hex and GalNAc. Since many products with the structure NeuAcα2→3Galβ1→3GalNAc (6→2αNeuAc) α1→O-bn have been obtained in the Benzyl-GalNAc experiment (FIG. 12), the possibility of having the same structure is believed to be high.

TABLE 1
Fragment ions of the product A2 produced by the MKN45 cell

| | | Observed | Calculated |
|---|---|---|---|
| Chemical species | | mass (m/z) | mass (m/z) |
| [M + Na]⁻ | (Primer + Hex + 2NeuAc) | 1269.88 | 1270.56 |
| [M-anNeuAc + Na]⁺ | (Primer + Hex + NeuAc) | 979.52 | 979.47 |
| [M-2anNeuAc + Na]⁺ | (Primer + Hex) | 688.54 | 688.37 |
| [M-NeuAc-anNeuAc-anHex + Na]⁺ | (Primer) | 526.48 | 526.32 |
| [M-NeuAc-anNeuAc-anHex + Na]⁺ | (anGalNAc + NeuAc) | 517.44 | 518.18 |
| [M-NeuAc-HexNAc-Thr-C₁₂H₂₄O + Na]⁺ | (anHex + NeuAc) | 476.35 | 477.16 |
| [M-2anNeuAc-anThr-C₁₂H₂₄O + Na]⁺ | (GalNAc + Hex) | 406.20 | 406.14 |
| [M-NeuAc-anHex-HexNAc-Thr-C₁₂H₂₄O—H + 2Na]⁺ | (NeuAc) | 354.29 | 354.11 |
| or [M-NeuAc-Hex-anHexNAc-Thr-C₁₂H₂₄O—H + 2Na]⁺ | | | |
| [M-NeuAc-Hex-HexNAc-Thr-C₁₂H₂₄O—H + 2Na]⁺ | (anNeuAc) | 336.26 | 337.10 |

Analysis of Compound A1

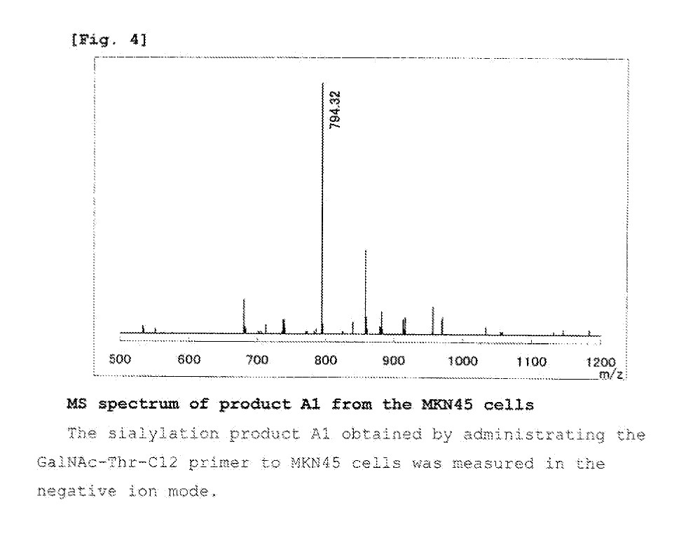

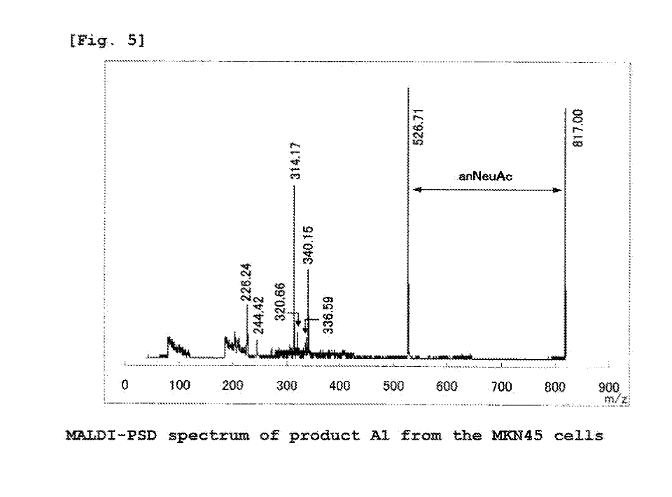

A peak at m/z=794.32 was obtained in negative ion mode (FIG. 4). The PSD spectrum (FIG. 5 and Table 2) revealed that this molecule had a structure wherein one NeuAc is linked to the primer. As it is believed that this may be a sialyl-Tn antigen (NeuAcα2→6GalNAc), sialidase was used to determine the mode of linkage for NeuAc.

TABLE 2
Fragment ions of the product A1 produced by the MKN45 cell

| | | Observed | Calculated |
|---|---|---|---|
| Chemical species | | mass (m/z) | mass (m/z) |
| [M + Na]⁺ | (Primer + NeuAc) | 817.00 | 817.42 |
| [M-NeuAc + Na]⁺ | (Primer) | 526.71 | 526.31 |
| [M-HexNAc-Thr-C₁₂H₂₄O—H + 2Na]⁻ | (anNeuAc) | 336.59 | 337.10 |
| [M-NeuAc-Hex-HexNAc-Thr-C₁₂H₂₄O + Na]⁺ | (anNeuAc) | 314.17 | 315.10 |
| [M-anNeuAc-anThr-C₁₂H₂₄O + Na]⁺ | (GalNAc) | 244.22 | 244.09 |
| [M-anNeuAc-Thr-C₁₂H₂₄O + Na]⁺ | (anGalNAc) | 226.24 | 227.09 |

### 2. Structural Analysis of Products of Sialylation by the HuH7 Cell

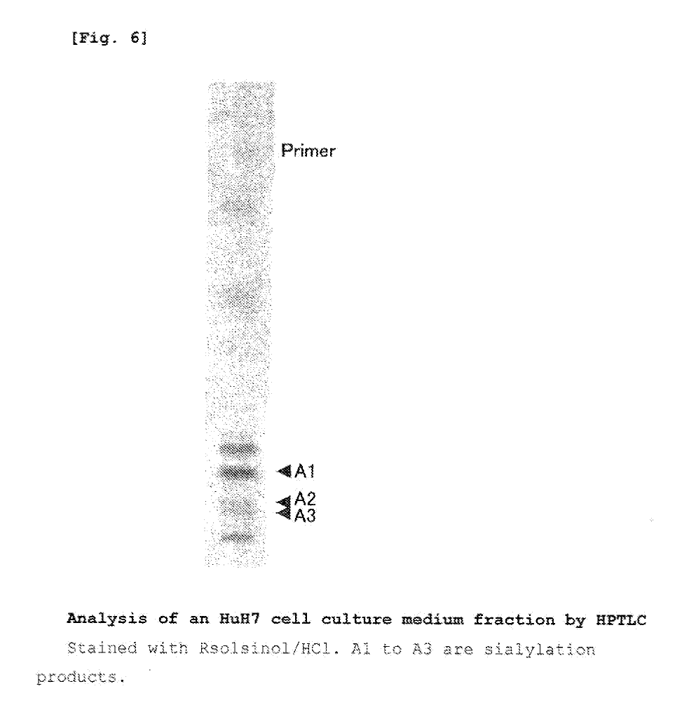

After administering GalNAc-Thr-C12 primer to HuH7 cells (human hepatoma cells), the medium fraction was purified, developed with HPTLC, and 3 bands of sialylation products were obtained (FIG. 6). The experiment was performed with the same method as for MKN45 cells. The three sialylation products were respectively designated A1 to A3.

Analysis of Compound A1

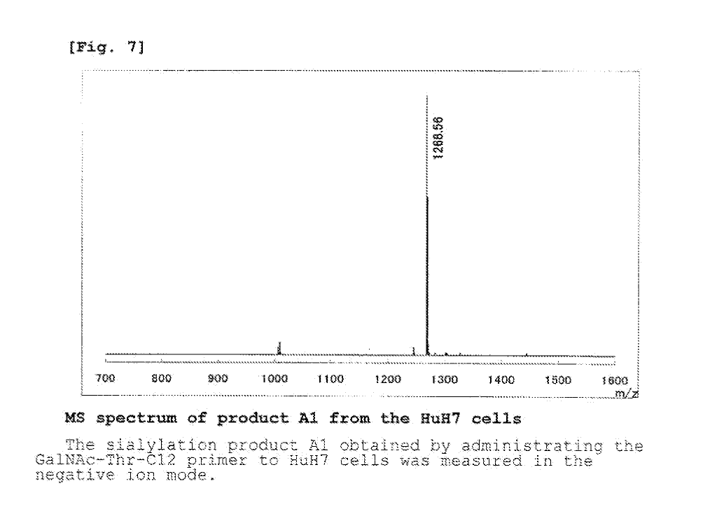

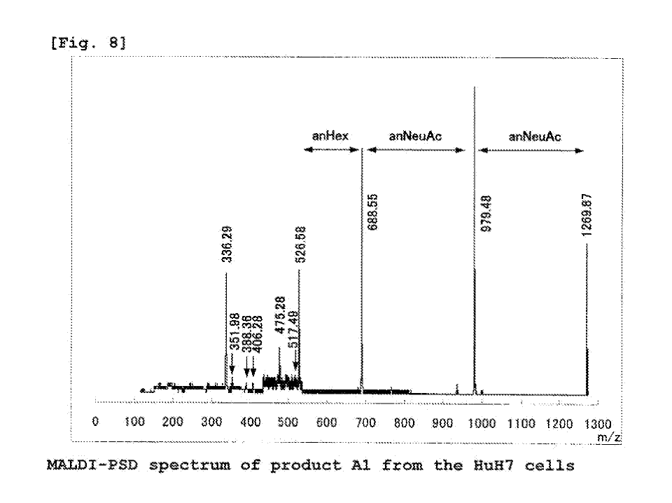

A peak at m/z=1268.56 was obtained by measurement in negative ion mode (FIG. 7). The PSD spectrum (FIG. 8 and Table 3) revealed that this molecule had a structure wherein one Hex and two NeuAc were linked to the primer, as was the case for A2 from MKN45 cells. In addition, from the fact that peaks are observed at m/z=517.49 and 475.28, one NeuAc is believed to be linked to each of Hex and GalNAc.

TABLE 3
Fragment ions of the product A1 produced by the MKN7 cell

| | | Observed | Calculated |
|---|---|---|---|
| Chemical species | | mass (m/z) | mass (m/z) |
| [M + Na]⁺ | (Primer + Hex + 2NeuAc) | 1269.87 | 1270.56 |
| [M-anNeuAc + Na]⁺ | (Primer + Hex + NeuAc) | 979.48 | 979.47 |
| [M-2anNeuAc-Na]⁺ | (Primer + Hex) | 688.55 | 688.37 |
| [M-NeuAc-anNeuAc-anHex + Na]⁺ | (Primer) | 526.58 | 526.32 |
| [M-NeuAc-HexNAc-Thr-C₁₂H₂₄O + Na]⁺ | (anHex + NeuAc) | 475.28 | 477.16 |
| [M-anNeuAc-anThr-C₁₂H₂₄O + Na]⁺ | (HexNAc + Hex) | 406.28 | 406.14 |
| [M-2anNeuAc-Thr-C₁₂H₂₄O + Na]⁺ | (anHexNAc + Hex) | 388.36 | 389.14 |
| [M-NeuAc-Hex-HexNAc-Thr-C₁₂H₂₄O—H + 2Na]⁺ | (anNeuAc) | 336.29 | 337.10 |

Analysis of Compound A2

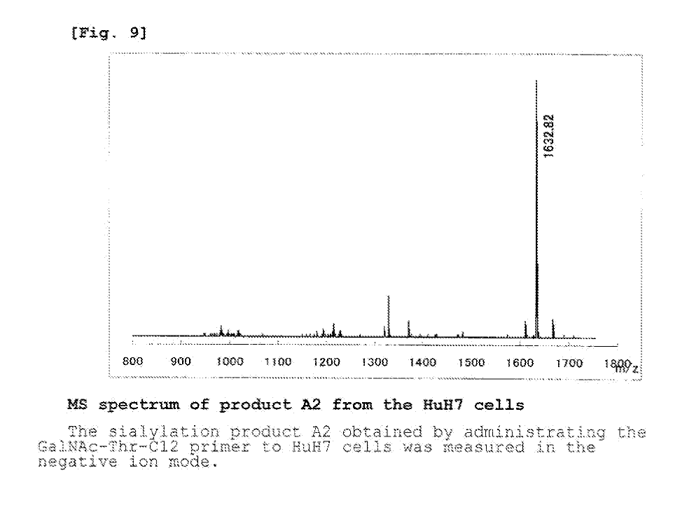

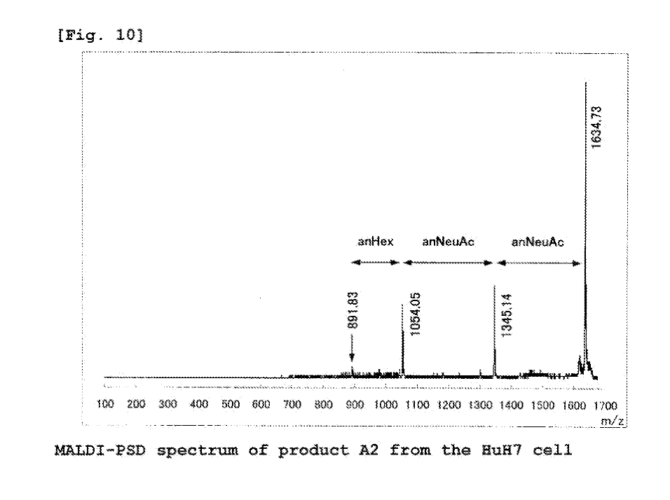

A peak at m/z=1632.82 was obtained in negative ion mode (FIG. 9). The PSD spectrum (FIG. 10 and Table 4) revealed that this molecule had a structure that contains one Hex and two NeuAc. In addition, since 891.83−526.32 (Primer+Na)=365.51, and this matches with the molecular weight of anHex+HexNAc or Hex+anHexNAc, there is a possibility that the structure comprises two Hex, one HexNAc, and two NeuAc linked to the primer (a product with such a structure has been also obtained in an experiment using Benzyl-GalNAc).

TABLE 4
Fragment ions of the product A2 produced by the MKN7 cell

| | Observed | Calculated |
|---|---|---|
| Chemical species | mass (m/z) | mass (m/z) |
| [M + Na]⁺ | 1634.73 | 1635.70 |
| [M-anNeuAc + Na]⁺ | 1345.14 | 1344.60 |
| [M-2anNeuAc + Na]⁺ | 1054.05 | 1053.51 |
| [M-NeuAc-anNeuAc-anHex + Na]⁺ | 891.83 | 891.45 |

1-29. (canceled)

30: A method for synthesizing an O-glycan-type sugar chain inside a cultured cell, comprising adding a saccharide primer to a cultured cell, wherein the O-glycan-type sugar chain comprises anyone from core type 1 to core type 8; and the saccharide primer comprises Formula (I): (G₁)ₓ(G₂)_{y}(G₃)_{z}-Aₘ-L-X, wherein G₁, G₂, and G₃ are independent monosaccharide residues with a pyranose ring, the monosaccharide residues being connected by α1-3, α1-6, α1-3 and α1-6 bonds; (G₁)ₓ(G₂)_{y}(G₃)_{z} is linear or branched, binding to Am through O-Glycan linkage; Aₘ is a sequence of 1 to 5 amino acids, and when there are a plurality of amino acids, the constituent amino acids may be identical or different; L is a linking group selected from the group consisting of —O—R—, —S—R—, —NH—R—, L binding to amino or carboxy group of Aₘ, R being an alkyl group, the alkyl group including a main carbon chain consisting of 6 to 20 carbons and comprising —S—S— or —NHCO— in place of —CH₂—CH₂—; X is a functional group selected from the group consisting of —N₃, —NH₂, —OH, —SH, —COOH, —OC(O)CH═CH₂, and —CH═CH₂; x, y, and z are independent integral numbers between 0 and 10, however, all of x, y, and z cannot be simultaneously 0.

31: The method of claim 30 wherein the cell is cultured using a high-density culture method.

32: The method of claim 30 wherein the cell is selected from the group consisting of an animal cell, a plant cell, an insect cell, and yeast.

33: The method of claim 30 wherein the cell contains a vector, a DNA coding for a glycosyltransferase having been integrated in the vector.

34: The method of claim 30 wherein (G₁)ₓ(G₂)_{y}(G₃)_{z} is GalNAc.

35: The method of claim 16 wherein Aₘ is Ser or Thr.

36: The method of claim 30 wherein X is —N₃.

37: A method for synthesizing an O-glycan-type sugar chain inside a cultured cell, comprising adding a saccharide primer to a cultured cell, wherein the O-glycan-type sugar chain comprises anyone from core type 1 to core type 8; and
the saccharide primer comprises GalNAcα1-Ser-O—(CH₂)ₙ—N₃ or GalNAcα1-Thr-O—(CH₂)ₙ—N₃, wherein, n is from 4 to 20.

38: The method of claim 37 wherein n is 12.

39: A method of claim 30, wherein the O-glycan-type sugar chain comprises one selected from a group consisting of Galβ1-3GalNAc; Galβ1-3(GlcNAcβ1-6)GalNAc; GlcNAcβ1-3GalNAc; GlcNAcβ1-3(GlcNAcβ1-6)GalNAc; GalNAcα1-3GalNAc; GlcNAcβ1-6GalNAc; GalNAcα1-6GalNAc; and Galα1-3GalNAc.
